(19) United States
(12) Patent Application Publication
(10) Pub. No.: US 2024/0423728 A1
(43) Pub. Date: Dec. 26, 2024

(54) METHODS AND APPARATUS FOR VALIDATION AND MODELING OF ROBOTIC SURGICAL TOOLS

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 18/782,896

(22) Filed: Jul. 24, 2024

Related U.S. Application Data
(63) Continuation-in-part of application No. PCT/IB2022/058978, filed on Sep. 22, 2022.
(60) Provisional application No. 63/303,008, filed on Jan. 25, 2022.
(60) Provisional application No. 63/567,659, filed on Mar. 20, 2024.

(51) Int. Cl. A61B 34/30; A61B 34/20; A61B 90/00

(52) U.S. Cl. CPC A61B 34/30; A61B 2034/2057; A61B 2034/2065; A61B 2090/3937

(57) ABSTRACT

A surgical robot a chassis includes a first surgical robotic arm and a reference device on a chassis. The first surgical robotic arm carries a surgical object, such as an implant, tool, or assembly, and the reference device carries indicia for identifying, sizing, calibrating, modeling, or verifying the surgical object. A controller kinematically positions the first surgical robotic arm to locate the implant, tool, or assembly in a predetermined orientation relative to the reference device in a surgical robotic coordinate space, and a camera is positioned to view the implant, tool, or assembly, when in proximity to the reference device.

### CROSS-REFERENCE TO RELATED APPLICATIONS

This application is a continuation-in-part of PCT application no. PCT/IB2022/058978, filed Sep. 22, 2022, which claims the benefit of U.S. Provisional No. 63/303,008 filed Jan. 25, 2022; this application also claims priority to U.S. Provisional No. 63/567,659, filed Mar. 20, 2024, the entire contents of each of which are fully incorporated herein by reference.

### BACKGROUND

Field. The disclosed technology relates generally to surgical robots and robotically controlled surgical procedures. More particularly, the disclosed technology relates to the identification, calibration, sizing, and verification of surgical tools and end effectors used in such procedures.

Robotic surgery and surgical robots are now in common use. A typical robotic surgical procedure requires deploying one or more surgical tools and end effectors on multiple surgical robotic arms using one or more robotically controlled cameras/sensors that may be located at varying distances and angulations from the surgical field. Accurate and precise placement of each of the tools and end effectors is necessary for the safe and successful completion of such robotic surgical procedures.

Positioning of the surgical tools and end effectors is often achieved “kinematically” where the surgical robot controls and tracks each tool and end effector based on the dimensions and angulations of each segment of a robotic arm in the robotic coordinate space. While kinematic tracking of the robotic arms can be very accurate, tracking of the tools themselves depends on accurately knowing the dimensions of the tool as well as the details of how the tool is attached to the robotic arm which can be difficult. For example, the working tips of tools held in grippers can extend unknown distances from the end of the surgical robotic arm that is being kinematically tracked, making fully automatic robotic surgical procedures difficult or impossible.

Because of that difficulty, many surgical robots can only be used with specific tools designed and certified for that particular robot. The dimensions and attachment details for approved tools will be known to the robotic controller, allowing the tools to be accurately and precisely deployed. There is often a mismatch and/or a lack of synchronization between many proprietary surgical robots and robotic tool sets available from other suppliers. Thus, many tool types available from multiple manufacturers which will be incompatible with specific surgical robots, and the need to obtain only approved tools is disadvantageous as it limits choice and can increase cost.

Robotic tool incompatibility is a particular problem in the spinal surgery market where a large variety of tools and implants are used in a wide variety of procedures. There are numerous manufacturers of tools and implants to be used in various spinal surgical procedures, with some tool types being available from only one or a few sources.

For these reasons, it would be desirable to provide surgical robotic systems which are capable of deploying a wide variety of surgical tools and end effectors available from a variety of manufacturers. It would be particularly desirable to enable a surgical robotic controller to identify, calibrate, size and/or verify surgical tools regardless of their source including those which are not designed to be compatible with a particular surgical robot.

Another challenge in robotic surgery is the misalignment of tools and end effectors that are sterilely packed with drivers, inserters, or the like. In some instances, even when the implant and driver are intended for the surgical robot being used, an implant assembly can become misaligned while packaged. Small misalignments can be difficult to detect, particularly when the implant assembly is unpackaged and mounted on a surgical robotic arm in a sterile zone prior to the procedure. This can occur, for example, when a screw, fusion cage, or other implant is pre-attached to a driver, inserter, or the like, to form a linear assembly. Even small misalignments (non-linear attachments) can be problematic and are difficult for the surgeon to visually detect.

A further problem arises when similar tools or implants have small differences in their specifications. For example, similarly sized pedicle screws can differ in pitch. While this is usually not a problem when the screw is advanced manually, it is a significant problem if the screw is driven by a robotically powered driver, The rates of advancement and rotation must be matched or the screw will not advance properly and, in the worst case, will core the bone (act as a drill). Thus, any mistakes in properly identifying the screw to the robot prior to deployment can have unfortunate consequences.

For all these reasons, there is a strong need for robotic systems and methods that can validate, e.g., identify, size, calibrate and/or verify, surgical tools and end effectors after the tools and end effectors have been mounted on a surgical robot arm of the robotic system. In some instances, the robotic systems and methods should function with both proprietary tools and end effectors designed to be compatible with a specific surgical robot as well as with generic tools and end effectors not designed to be compatible with the specific surgical robot. While human participation may sometimes be useful in such validation protocols. it will often be preferable to automate as much of the validation technology as possible. At least some of these objectives will be met by the technologies disclosed herein.

2. Background Art. Relevant commonly owned publications and applications include International Application No. PCT/IB2022/052297 (published as WO2022/195460); International Application No. PCT/IB2022/058986 (published as WO2023/067415); International Application No. PCT/IB2022/058972 (published as WO2023/118984); International Application No. PCT/IB2022/058982 (published as WO2023/118985); International Application No. PCT/IB2022/058978 (published as WO2023/144602); International Application No. PCT/IB2022/058980 (published as WO2023/152561); International Application No. PCT/IB2023/055047 (published as WO2023/223215); International Application No. PCT/IB2022/058988 (published as WO2023/237922); International Application No. PCT/IB2023/055439 (published as WO2024/089473); International Application No. PCT/IB2023/056911; International Application No. PCT/IB2023/055662; International Application No. PCT/IB2023/055663; International Application No. PCT/EP2024/052353; U.S. Provisional App. No. 63/524,911; and U.S. Provisional App. No. 63/532,753, the full disclosures of each of which are incorporated herein by reference in their entirety.

Systems for automated robotic inspection are described in WO2023/053130; WO2023/047405; WO2022/070186; WO2019/156783; and WO2016/083897, owned by Kitov Systems Ltd.

### SUMMARY

In a first aspect, the disclosed technology provides a surgical robot configured to validate a surgical object, such as a tool, implant, or assembly, after mounting on a surgical robotic arm but prior to deployment in a robotic surgical procedure. The surgical robot comprises a chassis, a first surgical robotic arm on the chassis configured to removably carry the implant or a tool, and a reference device configured for identifying, sizing, calibrating, modeling or verifying the surgical object. A controller is configured to kinematically position the first surgical robotic arm to locate the implant, tool or assembly in a predetermined orientation relative to the reference device in a surgical robotic coordinate space. In some examples, the predetermined orientation is selected to allow the implant or tool to be visually or optically characterized based upon indicia visible on the reference device.

As used herein, the term “implant” comprises any object or structure configured to be surgically implanted into the body of a patient. While the robotic methods, apparatus and systems of the disclosed technology are particularly useful in robotic orthopedic procedures intended for the implantation of screws, such as pedicle screws, fusion cages, interbody devices, and the like. They are also useful in other robotic surgical procedures for implanting a variety of other devices, including prosthetics, such as orthopedic implants, e.g., hip implants, knee implants, femoral heads, and the like; medication delivery devices; electrical stimulation devices, including electrodes, cannulas, etc.; diagnostic and monitoring devices; and the like.

As used herein, the term “tool” comprises any object or structure intended for use in any surgical implantation or other intervention where the tool is not itself implanted. For example, tools may comprise drivers or inserters for delivering implants, e.g., rotational drivers for inserting screws, inserters for holding a fusion cage or other implant as it is advanced into an insertion site, or the like. In other instances, the tool could be an interventional device, such as a grinder, an ablator, a cutter, a drill, a saw, a chisel, a cannula, an electrode, or the like where the device modifies or otherwise interacts with a patient's hard (bone) or soft tissue with implantation.

In many instances, the tools and implants will be combined into “assemblies” to facilitate robotic implantation. For example, an implant may be mounted on a driver, inserter or other active tool configured to allow or enable a robotic arm to perform a desired implantation, e.g., a rotational driver can hold a screw for implantation. In still other instances, the assembly may comprise an implant and a “holder,” e.g., a passive link or an interface intended primarily or solely to orient the implant at a desired distance and/or angulation relative to the surgical robot arm to facilitate implantation.

In many instances, the surgical object will be sterilely packed with the implant pre-attached to the tool. In such instances, the disclosed technologies can be used to verify that the attachment meets necessary specifications before the robotic procedure is started.

In other instances, the holder may be attached to an implant by immediately prior to the procedure in the sterile environment. In such instances, the disclosed technologies can be used to confirm that the attachment has been correctly performed.

Validation of the surgical tool, implant or assembly can take a variety of forms, including identifying, sizing, modeling, calibrating, modelling, and verifying the tool, implant or assembly.

Identification includes, for example, determining a specific model or serial number for a tool where the robotic controller can “look up” necessary specifications for that implant or tool. In other instances, the identification can be more generally directed at tool type or other features, e.g. by optically or otherwise comparing an image or profile of a or other surgical object held by a surgical robotic arm with an image of a tool type on the reference device.

Sizing includes determine a dimension of a tool, implant, or assembly or other surgical object, typically by comparing an image of the tool, implant, or assembly with a scale on the reference device. Dimensions include length, width, diameter, screw pitch, angle (such as a taper angle for a fusion cage), surface shape and structure, color, surface topology, e.g. special engraving, and the like.

Calibrating includes obtaining or confirming information regarding a surgical object, such as a tool, implant, or assembly and adjusting robotic system performance in some way in response to the information. For example, by determine screw pitch, a robotic controller can adjust rotation and advancement rates for the driver used to advance the screw into bone or other tissue.

Modeling includes digital imaging of features, shapes, and dimensions, and the like, of a surgical object, e.g., a tool, implant, or tool/implant assembly, based upon information derived by the methods of the disclosed technologies. The derived information will usually include, consist of, or consist essentially of image data obtained from a robotically controlled camera. For example, information may be obtained by comparisons with the reference device and can be combined with other optical or image information obtained by a system camera or other sensor to generate a two-dimensional or three-dimensional model of the surgical object. The model may be stored by the robotic controller and used in manipulating the modeled tool in a robotic surgical procedure. In particular, generic tools (tools for which the robotic controller does not have a pre-existing model) may be introduced to a surgical robot, modeled by the surgical robot to generate a two or three-dimensional model that allows the surgical robotic controller to kinetically manipulate the surgical robot arm which holds the tool in a subsequent robotic surgical procedure. Usually, the surgical object will be modeled together with a distal portion of the surgical robotic arm, grasper, or other end effector so hat that the robotic controller can kinematically tract many or all portions of the surgical object in the robotic surgical coordinate space.

Verifying includes using indicia on the reference tool to confirm that an assumed surgical object identification or specification is in fact correct prior to beginning a robotic surgical intervention. For example, the reference device may carry and/or receive a projected an image of the correct surgical object allowing a robotic or other camera to verify identity.

In specific instances, the predetermined orientation of the implant, tool or assembly relative to the reference device is generally perpendicular.

In other instances, the predetermined orientation of the implant, tool or assembly relative to the reference device is generally parallel.

The surgical robot of the disclosed technology may further comprise a second surgical robotic arm configured to carry the reference device, wherein the controller is configured to kinematically position the first and/or second surgical robotic arms to locate the reference device adjacent to the tool, implant, or assembly or other surgical object in the surgical robotic coordinate space.

In specific instances, the reference device of the surgical robots of the disclosed technology may be fixedly positioned on the chassis and the controller configured to kinematically position the first surgical robotic arm to locate the implant or tool adjacent to the fixed reference device in a surgical robotic coordinate space.

In other instances, the reference device of the surgical robots of the disclosed technology may be configured to be manually positioned in the surgical robotic coordinate space.

The surgical robot of the disclosed technology may further comprise a third surgical robotic arm configured to carry a camera, wherein the controller is configured to (1) kinematically position the first and/or second surgical robotic arms to locate the reference device in proximity to the implant or tool and (2) kinematically position the third surgical robotic arm to enable the camera to view the reference device and implant or tool.

In such instances, the controller may be configured to characterize the tool, implant or assembly by analyzing one or more images of the tool, implant or assembly and the reference device obtained from the camera while the reference device and the tool, implant or assembly are located in proximity.

In specific instances, the controller of the surgical robots of the disclosed technology may be configured to control deployment of the implant or tool in a robotic surgical procedure based upon the determined characteristic.

In specific instances, the determined characteristics of the surgical robots of the disclosed technology may comprise any one or more of dimensions, alignment, surface patterns, linearity, and screw pitch.

In specific instances, the indica of the surgical robots of the disclosed technology may comprise a scale.

In specific instances, the indica of the surgical robots of the disclosed technology may comprise a template.

In specific instances, the indica of the surgical robots of the disclosed technology may comprise a model,

In specific instances, the chassis of the surgical robots of the disclosed technology may comprise a mobile cart.

In other instances, the chassis of the surgical robots of the disclosed technology may comprise a surgical bed frame.

In a second aspect, the disclosed technology provides a surgical robot configured to validate (identify, calibrate, size and/or verify) a surgical object prior to deployment in a robotic surgical procedure. The surgical robot comprises a chassis carrying first, second, and third surgical robotic arms.

The first surgical robotic arm is configured to removably carry tool, implant, assembly, or other surgical object and the second surgical robotic arm is configured to carry a reference device, where the reference device is configured for identification, calibration, sizing, modeling, modeling, or verifying the tool, implant or assembly. The third surgical robotic arm is configured to carry a camera, and a controller is configured to kinematically position the first surgical and/or the second robotic arm to locate the surgical object in a predetermined orientation relative to the reference device in a surgical robotic coordinate space. The controller kinematically positions the camera so that the surgical object and the reference device are in a field of view of the camera. The controller is configured to (1) kinematically position the first and/or second surgical robotic arms to locate the reference device in proximity to the implant or tool and kinematically position the third surgical robotic arm to enable the camera to view the reference device and implant or tool. In this way, the controller can identify, calibrate, size and/or verify the surgical object based upon the images of the surgical object and indicia on the reference device from the camera.

In specific instances, the predetermined orientation of the surgical object and the reference device is generally perpendicular.

In other instances, the predetermined orientation of the surgical object and the reference device is generally parallel.

In specific instances, the controller is configured to characterize the tool, implant or assembly by analyzing one or more images of the tool, implant or assembly and the reference device obtained from the camera while the reference device and the tool, implant or assembly are located in proximity.

In specific instances, the controller is configured to control deployment of the implant or tool in a robotic surgical procedure based upon the determined characteristic. For example, the determined characteristic may comprise any one or more of dimensions, alignment, surface patterns, linearity, and screw pitch. In particular, the indicia may comprise any one or more of a scale, a template, a grid, or a model.

The indicia will typically be printed, inscribed, etched, or otherwise affixed to a surface of the reference device, and the reference devices can be interchanged when different indicia are needed. In other instances, as described in more detail below, the indicia may be projected onto a surface of the reference device allowing an unlimited library of indicia to be used with minimal system modification. Changes and updates can be implemented in the controller programming with little or no hardware changes.

In specific instances, the chassis may comprise a mobile cart.

In other instances, the chassis comprises surgical bed frame.

In a third aspect, the disclosed technology provides robotic surgical method comprising controlling movement of a first surgical robotic arm in a robotic coordinate space with a robotic controller to position an implant, tool, or assembly or other surgical object in proximity to, usually adjacent to, a reference device in a field of view of a camera. A camera images the reference device and the tool, implant, or assembly to generate image data for both the reference device and the tool, implant or assembly. The robotic controller validates, e.g., identifies, sizes, calibrates, models, or verifies, the tool, implant, or assembly by comparing the reference device image data with the implant or tool image data.

The methods of the disclosed technology may further comprise controlling movement of a second surgical robotic arm in the robotic coordinate space to position the implant, tool or assembly adjacent or otherwise in proximity to the reference device in a field of view of a camera, where the first surgical robotic arm carries the surgical object, such as an implant, tool, or assembly, and the second surgical robotic arm carries the reference device.

In some instances, the reference device is removably carried by the second surgical robotic arm.

In other instances, the reference device is affixed to the second surgical robotic arm.

In other instances, the first surgical robotic arm carries the implant or tool and the reference device is fixed relative to the first surgical robotic arm.

In other instances, the first surgical robotic arm carries the implant or tool the reference device is introduced manually.

The methods of the disclosed technology may further comprise controlling movement of a third surgical robotic arm to position the camera relative to the implant or tool and the reference device in the robotic coordinate space.

In some instances, controlling movement of at least one surgical robotic arm may comprises kinematically controlling said movement.

In some instances, controlling movement of each surgical robotic arm may comprises kinematically controlling said movement.

In some instances, the camera is not positioned by the robotic controller.

In some instances, the reference device and the tool, implant or assembly are imaged simultaneously with the camera.

In other instances, the reference device and the tool, implant or assembly are imaged sequentially with the camera. For example, the camera may be repositioned while imaging the reference device and the tool, implant or assembly with the camera.

In some instances, the reference device comprises indicia and the predetermined orientation aligns the indicia with the implant or tool in a field of view of the camera. For example, the indicia may comprise a scale, a template, or a shape.

In a fourth aspect, the disclosed technology provides a robotic surgical method for validating a tool, implant or assembly in a surgical robotic coordinate space. The method comprises controlling movement of a first surgical robotic arm with a robotic controller to position the tool, implant or assembly in the robotic coordinate space and controlling movement of a second surgical robotic arm with the robotic controller to position a reference device in the robotic coordinate space. The controller positions the first and/or second surgical robotic arms to locate the tool, implant, or assembly or other surgical object in proximity to the reference device, and a dimension or alignment of the tool, implant or assembly is determined by observing at least a portion of the tool while in proximity to the reference device.

In specific instances, the first and second surgical robotic arms are controlled by the robotic controller to engage a distal tip of the tool, implant or assembly against the reference device and wherein determining comprises the robotic controller calculating a length of the tool, implant or assembly based upon kinematically known relative positions of the first and second surgical robotic arms in the surgical robotic coordinate space. For example, the methods may further comprise controlling movement of a third surgical robotic arm with the robotic controller to position a camera in the surgical robotic coordinate space to observe when the distal tip of the tool engages the reference device.

In some instances, the first and second surgical robotic arms are controlled by the robotic controller to position a scale or template on the reference device adjacent to the tool.

In some instances, the methods further comprise controlling movement of a third surgical robotic arm with the robotic controller to position a camera in the surgical robotic coordinate space to observe the scale or template on the reference device in proximity to the tool. In such instances, the methods may further comprise reading a dimension of the tool, implant or assembly from the scale with the camera.

In some instances, the dimension may comprise a length, width, or diameter.

In specific instances, the tool, implant or assembly may comprise a screw and the dimension may comprise pitch.

In some instances, the tool, implant or assembly comprises a rotatable elongate body having an axis, said method further comprising rotating the tool, implant or assembly and observing the tip of the tool, implant or assembly with the camera to determine if said rotation causes the distal tip to process (wobble) about its axis. For example, the tip is\may be observed adjacent to a scale or template on the reference device to determine if tip precession exceeds an acceptable threshold.

In some instances, the tool, implant or assembly comprises an elongate body or assembly intended to have a straight axis, wherein observing comprises viewing the tool, implant or assembly adjacent to indicia on the reference device representing a straight template or line with the camera to determine if the tool, implant or assembly is straight.

In a fifth aspect, the disclosed technology provides a robotic surgical method for determining a scale factor between a surgical object and a reference device having a known dimension when viewed by a camera in a surgical robotic coordinate space.

The scale factor can be used by the surgical robotic controller to calculate an absolute dimension of the surgical device based on the apparent dimension as viewed by a surgical camera at a known location in a surgical robotic coordinate space.

The method comprises controlling movement of a first surgical robotic arm with the robotic controller to kinematically position the surgical object in the robotic coordinate space. A reference device and the camera are each provided at spaced-apart, known kinematic locations in the surgical robotic coordinate space, and the camera is used to image both the surgical object and the reference device. The robotic controller calculates the scale factor of the surgical object relative to the reference device based upon (1) the apparent dimensions of the reference device and the surgical object as viewed by the camera and (2) the kinematically known positions of the surgical object, the reference device, and the camera in the surgical robotic coordinate space.

In some instances, the surgical robotic controller further determines the actual dimension of the surgical object by multiplying the known dimension of the reference device by the scale factor. For example, the scale factor may comprise (be determined as) the multiplication product of (1) the ratio of the actual dimension and the apparent dimension of the feature and (2) the ratio of (a) the kinetically determined distance between the camera and the surgical object and (b) the kinetically determined distance between the camera and the reference device.

In some instances, the surgical object may comprise any one or more of a tool, an implant, and an assembly of a tool and an implant.

In some instances, providing the reference device at the known kinematic location in the surgical robotic coordinate space may comprise kinematically positioning a second robotic surgical arm which carries or comprises the reference device in the robotic coordinate space with the surgical robotic controller.

In some instances, providing the camera at the known kinematic location in the surgical robotic coordinate space may comprise kinematically positioning a third robotic surgical arm which carries or comprises the camera in the robotic coordinate space with the surgical robotic controller.

In some instances, the reference device may comprise a feature having known dimension(s) and the robotic controller determines an apparent dimension of the feature in an image from the camera.

In some instances, the feature may comprise any one or more of a scale, markings, indicia, and a profile.

In some instances, the reference device and the surgical object are imaged simultaneously by the camera.

In some instances, the reference device and the surgical object are imaged sequentially with the camera.

In a sixth aspect, the disclosed technology provides a method for robotically verifying that an actual surgical object, e.g., the specific surgical object that has been introduced to a surgical robot, is the surgical object that was intended to be introduced. Many tools, implants, and assemblies look similarly and can be confused through inattention.

The verification methods of the disclosed technology may comprise controlling a first surgical robotic arm with a robotic controller to position an actual surgical object in a robotic surgical field. Typically, but not necessarily, the actual surgical device will have a digital image available from a manufacturer, but in other cases, suitable images of specific surgical objects can be scanned into a surgical robot and stored in the controller or elsewhere.

The robotic controller further controls a second surgical robotic arm to position a reference device comprising an image projection region in the robotic surgical field adjacent to the actual surgical object.

An available or pre-scanned image of the intended surgical object is projected onto the image projection region, and an appearance of the actual surgical object is compared with the image projected on the image projection region to determine whether the appearance and the image are the same. If they are the same, the identity of the surgical object has been verified and the robotic surgical procedure can proceed.

In specific instances, projecting the image comprises controlling a third surgical robotic arm with the robotic controller to position a projector at a location selected to allow the projector to project the image on the image projection region.

In specific instances, comparing the appearance of the actual surgical object with the image projected on the image projection region comprises viewing both the actual surgical object and the image of the intended surgical object with a camera.

In some instances, the camera can be supported on the third surgical robotic arm which carries the camera. In other instances, the camera is supported on a fourth surgical robotic arm. In still further instances, the camera may be located outside of the robotic surgical field and located on a separate stand or other support.

In some instances, the appearance of the actual surgical object and the projected image are compared automatically by the controller. In other instances, the appearance of the actual surgical object and the projected image are compared visually by a user.

In a sixth aspect, the disclosed technology provides a system for controlling movement of a surgical tool in a robotic surgical coordinate space. The system is configured to scan a “generic” surgical tool, e.g., a tool which may be previously unknown to the surgical robotic system. By scanning the surgical tool, the robotic system controller can “model” the tool sufficiently to allow the controller to manipulate the tool in the system's surgical robotic coordinate space to perform a desired robotic surgical procedure. In this way, surgical robotic systems which had heretofore been limited to using previously characterized tools from a limited inventory can use many other tools as desired by each user. It is particularly advantageous that any tool can be loaded into the surgical robotic system immediately prior to the surgery and modeled for immediate use in the procedure. While some tool models may not meet minimum specified requirements for a particular surgical robot or procedure, the controller can alert the user and a different tool be selected.

This surgical robotic system comprises a central control unit and at least three robotic arms mounted on a single rigid chassis, allowing kinematic tracking and control of the robotic surgical arms, either with or without supplemental optical tracking and control. At least a first robotic arm is configured to carry a camera, at least a second robotic arm is configured to carry a calibration element, and at least a third robotic arm is configured to carry a surgical tool. The central control unit is configured to (1) scan the surgical tool and the calibration element with the camera to generate a three-dimensional model of the surgical tool and (2) control movement of the at least the third robotic arm in the robotic surgical coordinate space based on the generated three-dimensional model of the surgical tool.

In specific instances, the three-dimensional model includes at least at least one of length and diameter.

In specific instances, the control unit kinematically controls movement of all three surgical arms in a robotic coordinate space.

In specific instances, the single rigid chassis is mobile.

In specific instances, the central control unit is configured to bring the surgical tool into proximity of the calibration element prior to scanning by the camera to generate the three-dimensional model.

In specific instances, generating the three-dimensional model is based at least in part on the kinematically tracked positions of the robotic arms.

In specific instances, generating the three-dimensional model is based at least in part on an image produced by the camera.

In a seventh aspect, the disclosed technology provides a method for robotic control of a generic surgical tool in a robotic coordinate space. The method comprises robotically controlling the positions of a camera, a calibration element, and the generic surgical tool in the robotic surgical coordinate space so that the camera is positioned to view the generic surgical tool while in proximity to the calibration element. The camera scans the surgical tool and the calibration element with the camera to generate a three-dimensional model of the surgical tool, and movement of the surgical tool in the robotic surgical coordinate space is controlled to perform the robotic surgical procedure wherein movement control accommodates the generated three-dimensional model of the surgical tool.

In specific instances, the three-dimensional model includes at least at least one of length and diameter.

In specific instances, the robotically controlling the positions of the camera, the calibration element, and the generic surgical tool in the robotic surgical coordinate space comprises kinematically controlling movement of the first, second, and third robotic surgical arms in the robotic coordinate space.

In specific instances, the single rigid chassis is mobile.

In specific instances, the central control unit is configured to bring the surgical tool into proximity of the calibration element prior to scanning by the camera to generate the three-dimensional model.

In specific instances, generating the three-dimensional model comprises kinematically tracked positions of the camera, the calibration element, and the generic surgical tool.

In specific instances, generating the three-dimensional model comprises processing an image produced by the camera.

Further embodiments and implementations of the robotically controlled surgical systems of the disclosed technologies may comprise a centrally coordinated and synchronized robotic system for spinal surgery applications that allows for the precise sizing, positioning and calibration of robotic tools and implants. The robotic system may be mobile and portable. The system may comprise multiple robotic arms that each can hold at least one end effector, camera or navigation element for use in a spinal surgery procedure. The end effectors may include drilling tools or tool positioning elements. The cameras and navigation elements may provide guidance for the movement of the robotic arms and deployment of the end effectors and tools. Multiple cameras and navigation elements may be used to provide a diversity of navigation information. The robotic arms of the disclosed technology, while mounted on a single chassis, may have first joints that are spaced further apart than on multi-arm systems such as the da Vinci by Intuitive. Spaced apart robotic arms may provide for greater reachability and maneuverability and for the application of greater force and leverage to various surgical tasks, among other advantages.

The synchronized movement of the robotic arms may be augmented by the interaction of the navigation cameras/sensors with active or passive markers that may be placed at the beginning or during the procedure. The movement of the robotic arms may be synchronized by a central control unit from a single base that knows where the arms are based upon prior calibration of all arms. This robotic synchronization may be augmented by navigation cameras and markers. The single base can take the form of a rigid chassis that may optionally enclose the robotic arms in a retracted configuration. The base may be portable and can be moved in and out of the surgical field by, for example, being positioned under the surgical table and then being removed at the end or during the procedure.

Also disclosed are a system and method for a synchronized and coordinated robotic system that can be adapted to work with any tool, implant, assembly or other surgical object system. Disclosed is a mobile robotic system with multiple robotic arms that may be synchronized with each other and share the same rigid base or chassis.

In a surgical robotic procedure, such as a spinal surgery robotic procedure, the various robotic arms are synchronized with the bony anatomy through the use of appropriate imaging modalities (e.g., X-ray, CT, MRI etc.) and the use of markers as described herein that may be placed on the bony anatomy, on the soft tissue, or on the robotic arms or robotic tools themselves. The synchronized and coordinated robotic arms may then be used for the sizing and calibration of robotic tools or implants to be used in the surgical procedure according to the following details.

In some embodiments, the robotic system has at least two synchronized and coordinated robotic arms that are based in a common rigid chassis that is portable and that may be positioned under the surgical table for use during the surgical procedure. The movement of the robotic arms may be coordinated by a central control unit that is located within the rigid chassis. The central control unit may be able to gather navigation information provided by one or more cameras and by optionally placed markers on the soft tissue and/or bony anatomy of a patient. In this representative embodiment, one robotic arm holds a navigation camera/sensor that is able to track the patient with reference to the markers or other objects in the relevant space. A second robotic arm may hold a purpose-built calibration tool that has a set of dimensions that are known to the software of the robotic system. This calibration tool may also be part of the second robotic arm, but more flexibility may be provided by configuring the calibration tool to be detachable from the second robotic arm. In another embodiment, this calibration tool may be hand-held, thus two robotic arms may suffice for this purpose. A third robotic arm may then hold an end effector that can be configured to be adjustable, robotically or manually, and hold a large variety of surgical tool diameters, thus allowing the robotic system to accommodate the full range of diameters of robotic tools that can be found on the surgical market.

The tool can be placed inside this end effector manually by a surgeon or can be automatically robotically gripped by the robotic arms themselves. Once the tool is placed in the end effector, it can be brought in contact with the calibration device that is held by the second robotic arm or hand-held by the user. Held in this configuration, the tool can also be imaged and/or scanned by the navigation camera held by the first robotic arm and/or by another imaging device that is present in the operating room The first robotic arm holding the camera/sensor can now scan the tool and take multiple images of it from various positions (which are known to the central control unit). In this regard, several attributes of the robotic tool can be known, such as its diameter, its length and its three-dimensional model. These attributes can be provided to the central control unit of the robotic system and, taken together with the robotic system knowing the position of its robotic arms due to the imaging and sensing information provided by the cameras and sensors, the tool of known dimensions can be placed anywhere in the three-dimensional space of the surgical field that is in the view of the robotic system. The tool is thus calibrated, regardless of its manufacturer or precise design, allowing for the use of a very wide range of tools with a single robotic system. This genericizes the robotic system in terms of its tool compatibility, making it much more compelling to the surgeon as it allows for the use of tools that are precisely suited to the particular patient and surgical procedure. In this regard, the surgeon can choose what is best for the patient/procedure, rather than being limited by compatibility of a particular suite of tools or implants with the particular surgical robot being used at their hospital.

The sizing and calibration of tools and implants by two or three robotically coordinated arms can be done intraoperatively and in sterile environment. As explained before there are in the market numerous implants and tools sets but few of them can be used in every surgery (sometimes without prior planning due to unforeseen changing surgical requirements). Also, specifically in spine surgery, every tool set, contain large number of different tools (screwdrivers, taps etc.), all sterile and ready for surgery. Many times, these tools are being assembled by the nurse/doctor only intraoperatively when sterile together from several different components together with the implant to a complex mechanism which means it is impossible to size and/or calibrate them while being fully assembled before the surgery when non-sterile. The disclosed robotic system and calibration method allows for tool sizing and calibration for any random tool with or without the implant attached to it while being sterile during the surgery under sterile conditions without adding time or discomfort to the surgical staff. The disclosed approach is differentiated from other known solutions primarily because it can calibrate and size any surgical tool, such as a screwdriver and implant to be used in spinal surgery, prior but most importantly during the surgical procedure. This accommodates the widest possible ranges of surgical procedures since it allows for the calibration and sizing to be done intraoperatively. Thus, even if the surgical approach and tool set changes during the surgery, a new tool set can be calibrated and sized and introduced seamlessly into the surgical workflow. In previous approaches, tool sets can only be sized and calibrated by way of tool manufacturers placing proprietary navigation markers on the tools that are then recognizable by the robotic system that the tool set is designed to be used with. Thus, the use of the full range of tool sets with a generic robotic system is effectively impossible according to the current state of the art. This problem is solved by providing the current system for calibrating and sizing any tool set without the need for proprietary navigation markers.

All of these needs and elements may benefit tremendously from the central coordination and synchronized control of the inventive single-cart, multi-arm, non-teleoperated robotic system. Based on the placement of appropriately sized markers and the placement of navigation cameras at an appropriate distance and orientation to the target anatomy and the markers, movement of the robotic arms carrying end effectors, cameras and tools can be coordinated to provide for a safe and precise robotic spinal surgical procedure.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a robotic tool validation apparatus holding a surgical tool in a tool holder in proximity to a reference device in view of a camera, in accordance with some embodiments.

FIG. 2 is a detailed, enlarged view of the tool and tool holder of FIG. 1, in accordance with some embodiments.

FIG. 2A is a cross-sectional view taken along line 2A-2A of FIG. 1, in accordance with some embodiments.

FIG. 3 is a top view of a reference device configured to be removably mounted on a surgical robot arm and including a scale for validating a surgical tool, in accordance with some embodiments.

FIG. 4 illustrates mounting of the reference device of FIG. 3 on a distal interface end of a surgical robot arm, in accordance with some embodiments.

FIG. 5 illustrates a robotic validation system including first and second surgical robot arms and a camera being used to verify alignment of an assembly of a pedicle screw and a rotatable screwdriver in accordance with an embodiment of the disclosed technology.

FIG. 6 is a detailed view of the robotic validation system of FIG. 5 showing a distal tip of the pedicle screw adjacent to a scale on an upper surface of the reference device, in accordance with some embodiments.

FIGS. 7A and 7B compare the results of rotating a properly aligned screw and screwdriver assembly (FIG. 7A) and a misaligned screw and screwdriver assembly (FIG. 7B), in accordance with some embodiments.

FIG. 8 illustrates a reference device comprising a scale used to determine thread pitch on a pedicle screw, in accordance with some embodiments.

FIG. 9 illustrates a robotic arm arrangement which holds a camera, a reference device, and a surgical object and which is useful for determining a dimensional scaling factor in accordance with the disclosed technology.

FIG. 10 illustrates a robotic arm arrangement which holds an assembly of a fusion cage and an inserter in proximity to a reference device having an image projection region in the robotic surgical field, in accordance with some embodiments.

### DETAILED DESCRIPTION

With reference now to the figures and several representative embodiments, the following detailed description is provided.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

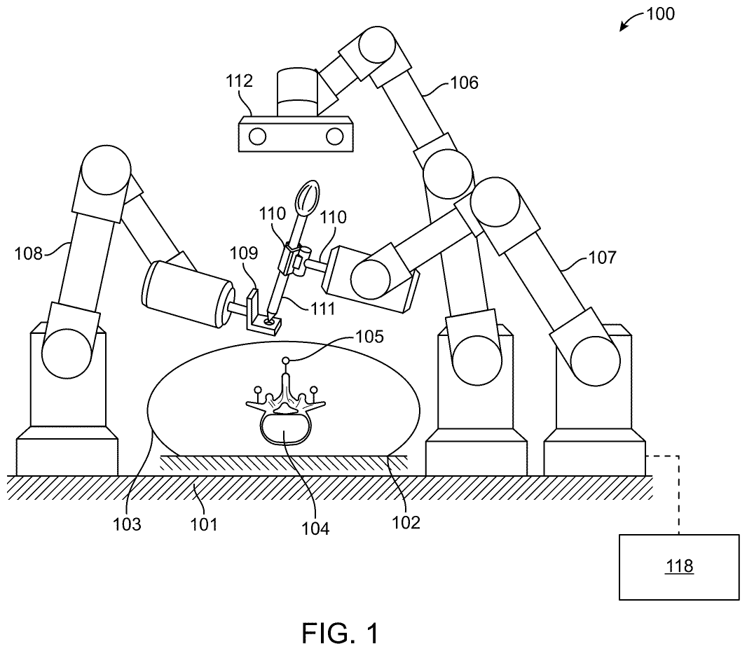

An exemplary robotic surgical system 100 intended for use in robotic spinal surgery is shown in FIG. 1, in accordance with some embodiments. The robotic surgical system 100 comprises a chassis 101. The chassis 101 may typically be a single, rigid and provides a base or platform for three robotic arms 106, 107 and 108 that are placed relatively far apart on the chassis 101, typically approximately one meter apart, thus allowing for desirable attributes such as reachability, maneuverability and ability to apply significant force. The chassis may be mobile, e.g., being in the form of a mobile cart as described in commonly owned International Application No. PCT/IB2022/052297 (published as WO2022/195460), previously incorporated herein by reference. In some embodiments and implementations, the surgical arms 106, 107 and 108 can be mounted on a base or other structure of a surgical table 102. The surgical robotic arms may be located on a stable platform that allows the arms to be moved within a common robotic coordinate system under the control of a surgical robotic controller 118.

The single, rigid chassis of the disclosed technology will usually comprise, consist of, or consist essentially of a single mobile cart, as disclosed for example in commonly owned International Application No. PCT/IB2022/052297 (published as WO2022/195460), the full disclosure of which has been previously incorporated herein by reference. In other instances, however, the single, rigid chassis may comprise separate modules, platforms, or components, that are assembled at or near the surgical table, as described for example in commonly owned PCT Application PCT/EP2024/052353, entitled Integrated Multi-Arm Mobile Surgical Robotic System, filed on Jan. 29, 2024, the full disclosure of which is incorporated herein by reference in its entirety. The single, rigid chassis may provide a stable base for all the surgical arms so that the surgical arms may be accurately and precisely kinematically positioned and tracked by the surgical robotic controller in a single surgical robotic coordinate space.

The chassis 101 of the robotic surgical system 100 is typically configured to be temporarily placed under the surgical table 102 when performing the robotic surgical procedure, allowing the robotic surgical system 100 to be stored remotely before and after the procedure. The robotic arms 106, 107 and 108 may optionally be configured to be retracted into the chassis 101 of the robotic surgical system, allowing the system to be moved into or out of the surgical field in a compact configuration.

As shown in FIG. 1, a patient 103 is positioned on the surgical table 102, and an element of the patient's spinal bony anatomy of interest, such as a vertebra 104, is exposed. A marker 105 may optionally be placed on the vertebra 104 or on another portion of the patient's bony anatomy or soft tissue.

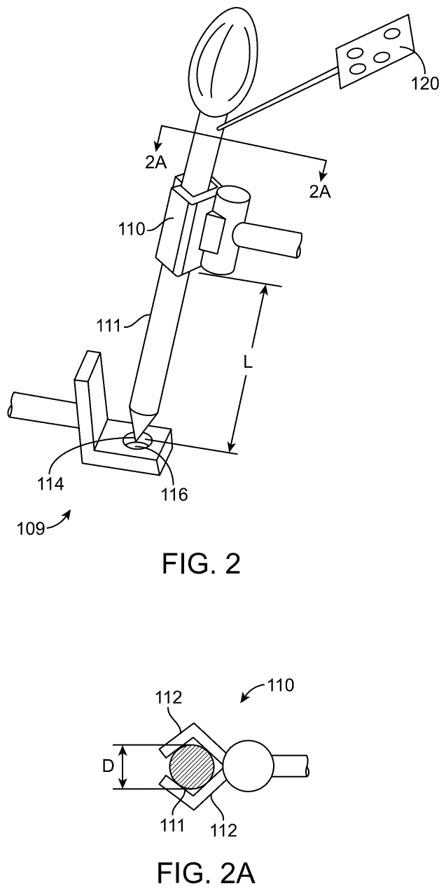

With further reference to FIGS. 1, 2 and 2A, the first robotic arm 106 may hold a navigation camera 112, in accordance with some embodiments. The second robotic arm 107 may hold a gripper 110 configured to removably hold an elongate tool 111, as shown in the detailed views of FIGS. 2 and 2A. The third robotic arm 108 may hold a reference device 109 in the form of an L-shaped bracket having a divot 116 or other marker or target on an upper face thereof.

Each of the camera 112, the gripper 110, and the reference device 109 may be removably of fixedly mounted or attached to a distal end of the associated surgical robotic arm. In some embodiments, at least the gripper 110 and the reference device 109 can be removably mounted so that the robot 100 can be used in other procedures which do not need the validation capabilities of the disclosed technology.

The surgical tool 111 may then be placed in the gripper 110 so that a tip 114 of the tool 111 is positioned against the divot or marker 116 of the reference device 109. The gripper 110 may have a fixed or an adjustable gripping diameter. As illustrated, the gripper 110 may include a pair of fixed jaws 112 sized to hold a tool 111 having a cylindrical tool shaft with a diameter D. In some embodiments, the gripper 110 can have an adjustable gripping diameter or width, as described in in commonly owned International Application No. PCT/IB2023/055047 (published as WO2023/223215), previously incorporated herein by reference. In still other instances, the gripper 110 may comprise a sleeve with a mechanism that can provide variable diameters depending on the tool 111 being used (not illustrated).

As illustrated, a gripper 110 having a width which accommodates a tool 111 having a specific shaft diameter D diameter may be selected from an inventory or library of differently sized grippers and be attached to the distal end of the surgical arm 108. In exemplary embodiments, the tool 111 can be advanced into the gripper 110 with a snug fit until the distal tip 114 reaches a stopping point defined by the divot 116 on the reference device 109. Alternatively, the tool 111 can be inserted into the gripper 110 to any desired depth and the surgical robotic arm 108 then moved to engage the divot 116 against the distal tip 114. In either case, the robotic controller 118 can sense or know the penetration depth of the tool 111 based upon the kinematically tracked positions of the surgical robotic arms 107 and 108. Optionally, the tool 111 can be releasably immobilized in the gripper 110 so that there is no slippage.

In further embodiments, a navigation marker 120 can be provided on the tool 111 to facilitate optical tracking by camera 112 or another camera or sensor. In some embodiments, the robotically manipulated camera 112 can be used for tracking as well as imaging the tool 111 and the reference device 114. In some instances, the robotically supported camera 112 can be kinematically tracked by the robotic controller 118, allowing the marker 120 and tool 111 to be tracked based on the previously established kinematic relationship between the surgical robotic arm and the tool 11 and tool tip 114.

In other embodiments, the marker 120 can be tracked in the robotic coordinate space by a remote navigation camera or sensor (not illustrated).

As described thus far, the camera 112 can be supported by surgical robotic arm 106 and positioned by the robotic controller 118. In other instances, a local navigation or tracking camera can be hand-held by a user.

Once the tip 114 of the tool 111 engages the divot 116 on reference device 109, a variety of tool characteristics can be determined.

In a first instance, the controller can determine a diameter or other width of the tool 111 based upon a width adjustment of the gripper and/or the size of gripper selected to accommodate the tool 111.

In a second instance, the controller can calculate a length L of the tool 111 based upon a kinematically established distance between the gripper 110 and the reference device 109. That is, by robotically positioning the surgical robotic arms 107 and 108 to locate the tool 111 and reference device 109 as shown in FIG. 2, the distance been a lower end of the gripper and the divot 116 on the reference device is known, which distance is predictive of the length L of the tool 111 which extends downwardly from the gripper.

In addition to calculating a length of the tool 11 as just described, the camera 112 can take single or multiple images of the tool 111, allowing the controller to generate two- and three-dimensional models.

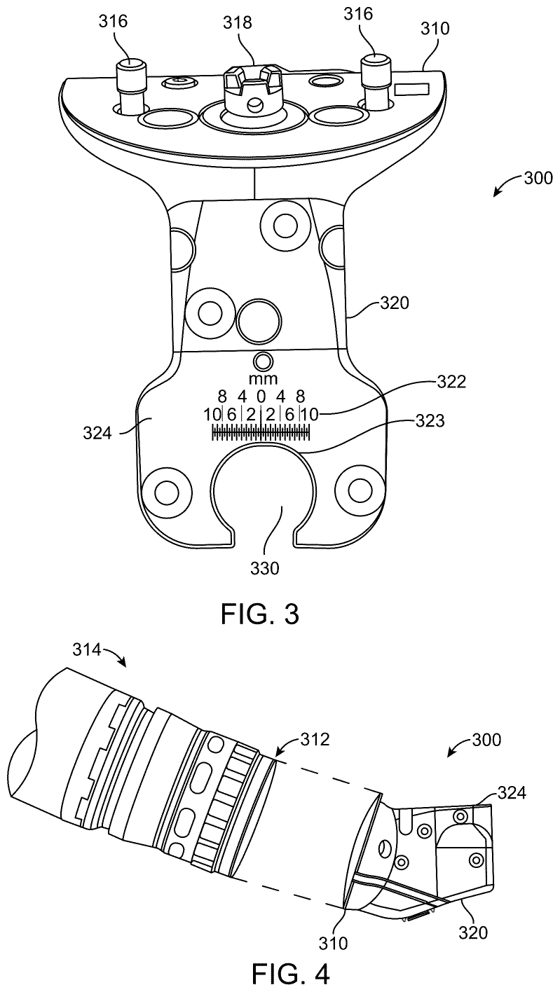

Referring now to FIGS. 3 to 7A and 7B, a reference device 300 intended for detecting precession or “wobble” in a tool assembly 310 will be described, in accordance with some embodiments. The reference device 300 may comprise a flange 310 configured to be removably attached to a distal end 312 of a first surgical robotic arm 314. The flange 310 may include connectors 316 and an interface element 318 which allow for such attachment. The connectors 316 may be screws that are used to assemble the gripper to the flange. The reference device 300 may include a lateral extension 320 having a scale 322 formed on an upper surface 324 thereof. The lateral extension 320 may also have a cut out 330 formed in its distal end.

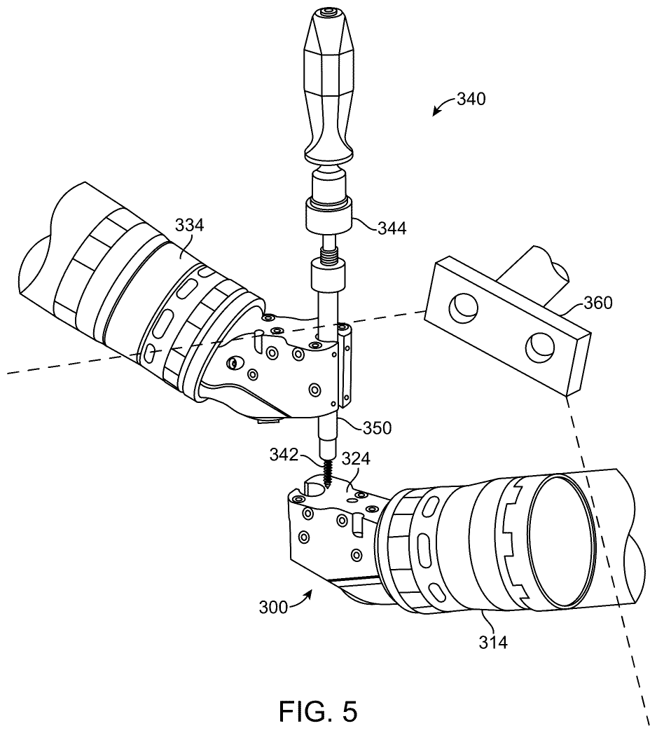

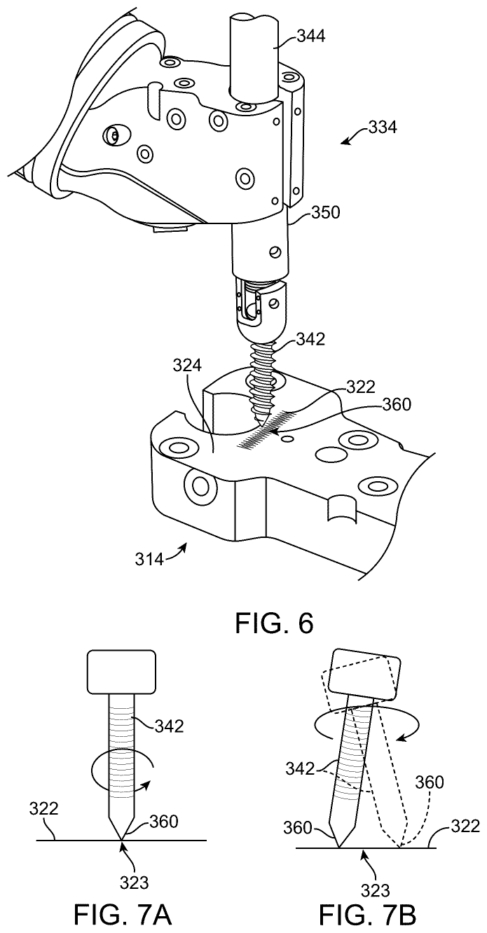

As shown in FIGS. 5 and 6, a second surgical robotic arm 334 may support a pedicle screw assembly 340 over the upper surface 324 of the lateral extension 320 of the reference device 300 which is supported by the first robotic arm 314, in accordance with some embodiments. The pedicle screw assembly 340 may include a pedicle screw 342 axially joined to a screwdriver 344, typically being pre-attached and sterilely packaged at the manufacturer. As illustrated, the pedicle screw assembly 340 may be rotationally inserted through a cannula 350, and the first and second surgical robot arms 314 and 334 may be kinematically positioned by a robotic controller so that a distal tip 360 of the pedicle screw 322 is located directly over a midpoint 323 of the scale 322.

As show in in FIGS. 7A and 7B, the screwdriver 344 can be rotated to cause the pedicle screw 342 to rotate over the midpoint 323 of the scale 322, in accordance with some embodiments. When the pedicle screw 342 is linearly attached to the screwdriver 344, e.g., the axis of the screwdriver is aligned with the axis of the pedicle screw, the distal tip 360 of the pedicle screw can remain stationary over the midpoint of the scale, as shown in FIG. 7A, and the connection of the assembly has been verified to be correct. In contrast, if the axis of the pedicle screw 342 is misaligned with the axis of the screwdriver 344, the tip 360 of the pedicle screw can process or “wobble” about the midpoint 323, as shown in FIG. 7B. In such cases, the alignment of the pedicle screw assembly 340 can be corrected or the entire assembly can be replaced with a new, sterile assembly. Use of the scale 322 also allows the extent of non-linearity to be measured. In most instances, some tip precession will be acceptable, e.g., ±1 mm for a 3 cm to 6 cm pedicle screw. The degree of precession can be determined visually by the user but will more usually be determined optically using the camera 360 and the robotic controller.

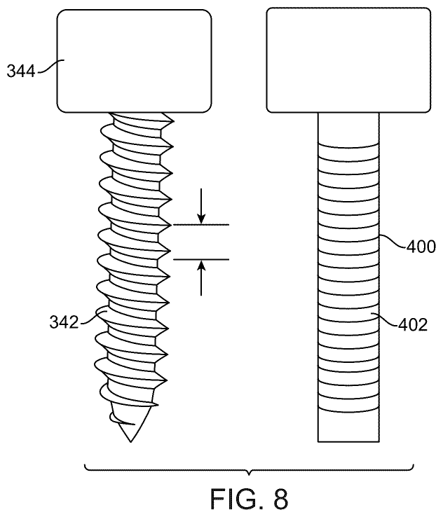

The validation features of the disclosed technology can also be used to measure or calibrate the pitch of a pedicle or other medical screw. As shown in FIG. 8, the pedicle screw 342 of FIGS. 5 and 6 can be robotically supported in a robotic coordinate space in proximity to a reference device 400 comprising a linear scale 402 having indicia corresponding to screw pitch, e.g., the distance between two successive screw threads, in accordance with some embodiments. While the pitch can be calculated in a variety of ways, one straightforward protocol is to use a scale 402 which is much finer that the expected screw pitch (multiple scale markings for each likely pitch distance) and to measure the distance between adjacent screw threads by optically viewing with the scale markings with the camera 360 and controller. single, rigid chassis.

In many cases, the reference devices of the disclosed technology may be used to determine an actual dimension of a surgical object held by a surgical robotic arm in a surgical robotic coordinate space. Often, a robotic controller can only determine an “apparent” dimension of a surgical object viewed by a robotic camera. Even when a distance between the camera and the viewed surgical object can be kinematically or otherwise determined, calculation of an absolute dimension can be difficult.

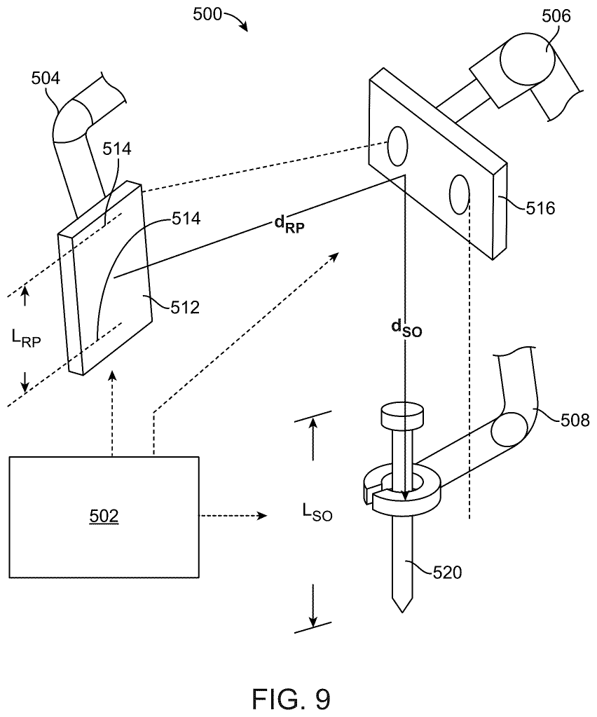

The disclosed technology provides a more accurate system and method for determining an absolute dimension as illustrated in FIG. 9, in accordance with some embodiments. A surgical robot 500 may include a robotic controller 502 which controls and kinematically tracks the positions of first, second, and third surgical robotic arms 504, 506, and 508, respectively. The first surgical robotic arm may carry a reference device 512 which has a known dimension which can viewed by a camera 516 carried by the second surgical robotic arm 506. In FIG. 9, the known dimension is the distance between two marker lines 514, but it also could be a length of the reference device, a distance between any two features on the surgical robotic arm itself or a tool or end effector carried by arm, or the like. In still other cases, the known dimension can be any other fixed distance which can be viewed by the camera at a kinematically known location in the surgical robotic coordinate space.

An actual dimension of a surgical object, such as a length LSO of a surgical probe 520 held by surgical robotic arm 580, can be determined by the robotic controller determining an apparent length of LSO-APP of the probe and an apparent length LRD-APP between the markers 512 and 514 on the reference device 512. “Apparent” length can mean the length as viewed by the controlled in an image taken by the camera 516. Such apparent lengths may be in arbitrary units as only their ratios will be used in calculating the actual length or other dimension. The actual length LSO-ACT of the probe 520 can be calculated as follows:

$${L}_{SO - ACT} = {L}_{RD - ACT} \times ( {L}_{RD - APP} / {L}_{RD - ACT} ) \times ( {d}_{SO} / {d}_{RD} )$$

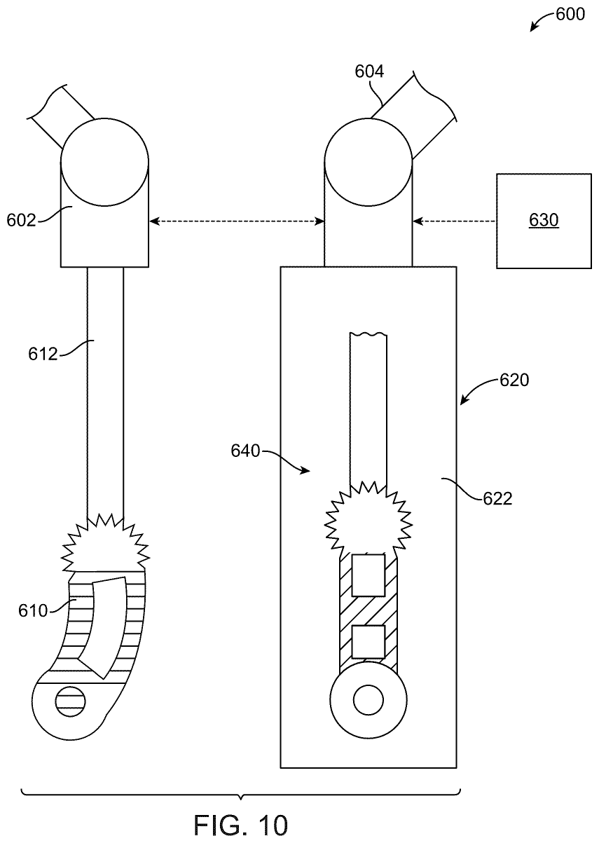

Referring now to FIG. 10, surgical robotic arms 602 and 604 of a surgical robot 600 (only partially illustrated) hold (1) an assembly of a fusion cage 610 and inserter 612 and (2) a reference device 620 having an image projection region 622, in accordance with some embodiments. Movements of the robotic arms 602 and 604 may be kinetically controlled by a surgical robotic controller 630 as described previously with respect to other embodiments. An image projector (not illustrated) may also be present and can be positioned by the robotic controller 630 to project an image 640 of the intended surgical object on the image projection region 622. The projector can typically be mounted on a separate robotic arm (not illustrated) and in some embodiments can be mounted on the same arm as a camera (not illustrated).

As illustrated in FIG. 10, in an example, the image 640 of the fusion cage and inserter may differ significantly from the appearance of the actual fusion cage and inserter, so the user can be alerted that the actual surgical object assembly that has been loaded onto the surgical robot 600 is probably not the intended assembly.

The surgical robotic arms 602 and 604 can be positioned so that the actual assembly of the fusion cage 610 and inserter 612 are held adjacent to the image projection region 622 so that a user can easily compare. In other instances, the image projection region 622 can be partially or fully transparent, allowing the projected image to be superimposed over the actual device in a chosen line-of-sight.

Comparisons can be done by direct visual inspection, but in other instances can be done by a robotic camera (not illustrated), either by the robotic controller automatically comparing the projected image with the appearance of the actual surgical object or by providing the actual and projected images on a display screen to allow the user to view.

In some instances, comparisons may be performed without the use of the separate reference device. For example, an outline or other image of an intended device can be projected directly onto the actual surgical object, allowing a visual or automatic comparison. In still other instances, the robotic controller could compare a digital representation with a digitized image of the actual surgical object without the need for projections. Such approaches, however, may be more difficult to implement and may not allow the user participation that is often desirable.

Use of the systems and methods of the disclosed technologies allows for validation of a wide variety of tools, implants and assemblies by a surgical robotic system, including identifying, sizing, modeling, calibrating, modelling, and verifying the tools, implants and assemblies by a surgical robot prior to commencing a surgical robotic procedure. The ability to validate both proprietary and non-proprietary tools, implants and assemblies allows a user to choose the best tool for any procedure, regardless of the manufacturer of the tool.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. A surgical robot configured to validate a surgical object after mounting on a surgical robotic arm but prior to deployment in a robotic surgical procedure, said surgical robot comprising:
a chassis;
a first surgical robotic arm on the chassis configured to removably carry an implant or a tool;
a reference device configured for sizing calibration, or verification of the surgical object; and
a controller configured to kinematically position the first surgical robotic arm to locate the implant or tool in a predetermined orientation relative to the reference device in a surgical robotic coordinate space;
wherein the predetermined orientation is selected to allow the implant or tool to be visually or optically characterized based upon indicia visible on the reference device.

2. The surgical robot of claim 1, wherein the predetermined orientation is generally perpendicular.

3. The surgical robot of claim 1, wherein the predetermined orientation is generally parallel.

4. The surgical robot of claim 1, further comprising a second surgical robotic arm configured to carry the reference device, wherein the controller is configured to kinematically position the first and/or second surgical robotic arms to locate the reference device adjacent to the surgical object in the surgical robotic coordinate space.

5. The surgical robot of claim 1, wherein the reference device is fixedly positioned on the chassis and the controller configured to kinematically position the first surgical robotic arm to locate the implant or tool adjacent to the fixed reference device in a surgical robotic coordinate space.

6. The surgical robot of claim 1, wherein the reference device is configured to be manually positioned in the surgical robotic coordinate space.

7. The surgical robot of claim 4, further comprising a third surgical robotic arm configured to carry a camera, wherein the controller is configured to (1) kinematically position the first and/or second surgical robotic arms to locate the reference device in proximity to the implant or tool and (2) kinematically position the third surgical robotic arm to enable the camera to view the reference device and implant or tool.

8. The surgical robot of claim 7, wherein the controller is configured to characterize the surgical object by analyzing one or more images of the surgical object and the reference device obtained from the camera while the reference device and the surgical object are located in proximity.

9. The surgical robot of claim 1, wherein the controller is configured to control deployment of the implant or tool in a robotic surgical procedure based upon the determined characteristic.

10. The surgical robot of claim 1, wherein the determined characteristic comprises any one or more of dimensions, alignment, surface patterns, linearity, and screw pitch.

11. The surgical robot of claim 1, wherein the indicia comprise a scale.

12. The surgical robot of claim 1, wherein the indicia comprise a template.

13. The surgical robot of claim 1, wherein the indicia comprise a model.

14. The surgical robot of claim 1, wherein the chassis comprises a mobile cart.

15. The surgical robot of claim 1, wherein the chassis comprises surgical bed frame.

16. A surgical robot configured to validate a surgical object prior to deployment in a robotic surgical procedure, said surgical robot comprising:
a chassis;
a first surgical robotic arm on the chassis configured to removably carry an implant or a tool;
a second surgical robotic arm on the chassis configured to carry a reference device, wherein the reference device is configured for robotic calibration, sizing, or verification of the surgical object;
a third surgical robotic arm on the chassis configured to carry a camera; and
a controller is configured to kinematically position the first surgical and/or the second robotic arm to locate the surgical object in a predetermined orientation relative to the reference device in a surgical robotic coordinate space and to kinematically position the camera so that the surgical object and the reference device are in a field of view of the camera to allow the controller to identify, calibrate, size and/or verify the surgical object based upon indicia visible on the reference device.

17. The surgical robot of claim 16, wherein the predetermined orientation is generally perpendicular.

18. The surgical robot of claim 16, wherein the predetermined orientation is generally parallel.

19. The surgical robot of claim 16, wherein the controller is configured to characterize the surgical object by analyzing one or more images of the surgical object and the reference device obtained from the camera while the reference device and the surgical object are located in proximity.

20. The surgical robot of claim 16, wherein the controller is configured to control deployment of the implant or tool in a robotic surgical procedure based upon the determined characteristic.

21. The surgical robot of claim 20, wherein the determined characteristic comprises any one or more of dimensions, alignment, surface patterns, linearity, and screw pitch.

22.-26. (canceled)

27. A robotic surgical method comprising:
controlling movement of a first surgical robotic arm in a robotic coordinate space with a robotic controller to position surgical object adjacent to a reference device in a field of view of a camera; and
imaging the reference device and the surgical object with the camera to generate image data for the reference device and for the surgical object;
wherein the robotic controller validates the surgical object by comparing the reference device image data with the implant or tool image data.

28.-101. (canceled)
